(19) United States
(12) Patent Application Publication
(10) Pub. No.: US 2010/0130367 A1
(43) Pub. Date: May 27, 2010

(54) Methods of Quantitatively Assessing Inflammation with Biosensing Nanoparticles

(75) Inventors: Sreekant Murthy, Sewell, NJ (US); Elisabeth S. Papazoglou, Yardley, PA (US); Kambiz Pourrezaei, Gladwyne, PA (US); Som Tyagi, Boothwyn, PA (US); Amolkumar Karwa, Paoli, PA (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Drexel University, Philadelphia, PA (US)

(21) Appl. No.: 12/307,901

(22) PCT Filed: Jul. 11, 2007
(86) PCT No.: PCT/US07/15748

§ 371 Date: Oct. 1, 2009

Related U.S. Application Data
(60) Provisional application No. 60/830,174, filed on Jul. 11, 2006.

(51) Int. Cl. C40B 30/00; G01N 33/573; G01N 33/536; G01N 33/53; C12Q 1/68

(52) U.S. Cl. 506/7; 435/7.1; 435/7.4; 435/7.2; 435/7.92; 435/6

(57) ABSTRACT

The present invention includes a method of using one or more biomarkers to identify individuals with inflammatory disease using Quantum Dots conjugated to targeting moieties that will specifically bind to biomarker proteins or nucleic acids encoding the biomarker, where detection of the biomarker is associated with the inflammatory disease.

### BACKGROUND OF THE INVENTION

Inflammatory Bowel Disease (IBD) encompasses two chronic, related inflammatory conditions, ulcerative colitis (UC) and Crohn's disease (CD). In addition, organs other than the intestinal tract can be involved by the underlying inflammation of IBD thus making IBD a multi-organ disease. As many as 4 million people (including one million Americans) worldwide suffer from a form of IBD. In the U.S. alone, IBD accounts for approximately 152,000 hospitalizations each year. The annual medical cost for the care of IBD patients in the United States is estimated at over $2 billion. When adjusted for loss of productivity, the total economic burden is estimated to be nearly $3 billion.

The diagnosis of IBD is rarely straightforward, involving an extensive process of examination and invasive testing, including biopsy during endoscopy. Even with these specialized studies, it is often still difficult to tell which type of IBD a person has, leading to a diagnosis of “indeterminate colitis” and rendering disease management more difficult. Since UC in particular is associated with a 35% higher risk of developing colorectal cancer than the general population, making a proper diagnosis is essential to good patient care.

While there is no medical cure for IBD, effective medical treatment is available which can calm the inflammation and relieve the symptoms of diarrhea, abdominal pain, and rectal bleeding. Since the disease tends to manifest itself with multiple attacks and remissions, continuous monitoring of patients is essential to provide the necessary medical treatment to reduce inflammation and prevent the development of clinical sequelae. Thus, there is a long-standing need for non-invasive diagnostic tools that are able to distinguish non-IBD symptoms from IBD, accurately distinguish UC from CD, and monitor disease progression, remission or relapse. The current invention fulfills this need.

### SUMMARY OF THE INVENTION

One embodiment of the invention comprises a method of identifying an inflammatory condition in a mammal, the method comprising obtaining a biological sample from the mammal, contacting the sample with a conjugate where the conjugate comprises a reporter component and an antibody that specifically binds to a biomarker, and determining whether the conjugate binds to the biomarker, where the binding of the conjugate is an indication that the mammal is afflicted with an inflammatory disease. By “specifically binds” is meant a molecule, such as an antibody, which recognizes and binds to a cell surface molecule or feature, but does not substantially recognize or bind other molecules or features in a sample.

In one aspect, the reporter component comprises a quantum dot. In another aspect, the reporter component comprises a magnetic nanoparticle. In yet another aspect, the reporter component comprises a magnetic quantum dot. In a further aspect, the mammal is a human. In still another aspect, the method comprises detecting two or more biomarkers in a biological sample. In another aspect, the biomarker is selected from the group consisting of an enzyme, an adhesion molecule, a cytokine, a protein, a lipid mediator, an immune response mediator, and a growth factor. In yet another aspect, the biomarkers of the invention are selected from the group consisting of myeloperoxidase (MPO), IL1α, TNFα, perinuclear anti-neutrophil cytoplasmic antibody (p-ANCA), anti-Saccharomyces cerevisiae antibody (ASCA), angiotensin converting enzyme, lactoferrin, C-reactive protein (CRP), and calprotectin. In still another aspect of the invention, the inflammatory condition or disease is selected from the group consisting of inflammatory bowel disease, ulcerative colitis, Crohn's disease, stroke, myocarditis, cardiovascular disease, acute coronary syndromes, acute myocardial infarction, pericarditis, periodontal disease, cancer, Alzheimer's disease, and autoimmune diseases. In a further aspect, the method comprises an immunoassay selected from the group consisting of Western blot, ELISA, immunopercipitation, immunohistochemistry, immunofluorescence, radioimmunoassay, dot blotting, and FACS. In yet another aspect, the method comprises a nucleic acid assay selected from the group consisting of a Northern blot, a Southern blot, in situ hybridization, a PCR assay, an RT-PCR assay, a probe array, a gene chip, and a microarray.

Another embodiment of the invention comprises a kit comprising a composition for detecting a biomarker in a biological sample obtained from a mammal, wherein the composition comprises at least one conjugate, further wherein the conjugate comprises a reporter component and an antibody that specifically binds to a biomarker, and instructional material for the use thereof.

In one aspect, the reporter component is at least one member selected from the group consisting of a quantum dot, a magnetic nanoparticle, and a magnetic quantum dot. In another aspect, the mammal is a human. In yet another aspect, the biomarker is selected from the group consisting of myeloperoxidase (MPO), IL1α, TNFα, perinuclear anti-neutrophil cytoplasmic antibody (p-ANCA), anti-Saccharomyces cerevisiae antibody (ASCA), angiotensin converting enzyme, lactoferrin, C-reactive protein (CRP), and calprotectin. In still another aspect, the antibody is bound to a substrate surface.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1C, is a series of images depicting MPO expression on Day 0 of the DSS model (Control). FIG. 1A is an image of a brightfield photomicrograph. FIG. 1B is a maximum projection image. FIG. 1C is an image of a photomicrograph of a section stained with hematoxylin and eosin.

FIG. 2, comprising FIG. 2A through FIG. 2D, is a series of images depicting MPO expression on Days 3 and 4 of the DSS model. FIG. 2A is an image depicting a maximum projection of MPO taken on Day 3. FIG. 2B is an image of a photomicrograph of a section stained with hematoxylin and eosin taken on Day 3. FIG. 2C is an image depicting a maximum projection of MPO taken on Day 4. FIG. 2D is an image of a photomicrograph of a section stained with hematoxylin and eosin taken on Day 4.

FIG. 3, comprising FIG. 3A through FIG. 3D, is a series of images depicting MPO expression on Days 6 and 7 of the DSS model. FIG. 3A is an image of MPO expression on Day 6. FIG. 3B is an image of a brightfield photomicrograph of a section stained with hematoxylin and eosin taken on Day 6. FIG. 3C is an image of MPO expression on Day 6. FIG. 3D is an image depicting MPO expression on Day 7.

FIG. 4, comprising FIG. 4A through FIG. 4C, is a series of charts depicting disease progression in the DSS model. FIG. 4A is a chart depicting disease activity index (DAI) as a function of time. FIG. 4B is a chart depicting fluorescence intensity as a function of time. FIG. 4C is a chart depicting fluorescence intensity as a function of DAI.

FIG. 5, comprising FIG. 5A through FIG. 5D, is a series of images depicting Day 3 of DSS feed. FIG. 5A is a maximum projection depicting 655QDs on Day 3. FIG. 5B is an image of a brightfield photomicrograph of a section stained with hematoxylin and eosin taken on Day 3. FIG. 5C is a maximum projection depicting 655QDs on Day 3 from another section of the same animal. FIG. 5D is a maximum projection depicting 655QDs on Day 3 from another section of the same animal.

FIG. 6, comprising FIG. 6A through FIG. 6D, is a series of images depicting Day 5 of DSS feed. FIG. 6A is a maximum projection depicting 655QDs on Day 3. FIG. 6B is an image of a brightfield photomicrograph of a section stained with hematoxylin and eosin taken on Day 5. FIG. 6C is a maximum projection depicting 655QDs on Day 3 from another section of the same animal. FIG. 6D is a maximum projection depicting 655QDs on Day 3 from another section of the same animal. Arrows indicate QDs.

FIG. 7, comprising FIG. 7A through FIG. 7F, is a series of images depicting QD labeling on Day 8 of DSS feed. FIG. 7A is a maximum projection depicting 655QDs on Day 8. FIG. 7B is an image of a brightfield photomicrograph of a section stained with hematoxylin and eosin taken on Day 8. FIG. 7C is a maximum projection depicting 655QDs on Day 8 from another section of the same animal. FIG. 7D is a maximum projection depicting 655QDs on Day 8 from another section of the same animal. FIG. 7E is a maximum projection depicting 655QDs on Day 8 from another section of the same animal. FIG. 7F is an image of a brightfield photomicrograph of a section stained with hematoxylin and eosin taken on Day 8.

FIG. 8, comprising FIG. 8A through FIG. 8C, is a series of charts depicting disease progression in the DSS model. FIG. 8A is a chart depicting disease activity index (DAI) as a function of time. FIG. 8B is a chart depicting fluorescence intensity as a function of time. FIG. 8C is a chart depicting fluorescence intensity as a function of DAI.

FIG. 9, comprising FIG. 9A through FIG. 9D, is a series of images depicting the specificity of the MPO conjugate in the DSS model. FIG. 9A is a maximum projection image of on Day 4 of DSS feed. FIG. 9B is an image of a brightfield photomicrograph of a section stained with hematoxylin and eosin taken on Day 4. FIG. 9C is a maximum projection image of on Day 6 of DSS feed. FIG. 9D is an image of a brightfield photomicrograph of a section stained with hematoxylin and eosin taken on Day 6.

FIG. 10, comprising FIG. 10A through FIG. 10D, is a series of images depicting IL1α, MPO and TNFα expression on Day 3 of DSS feed. FIG. 10A is an image depicting a maximum projection of IL1α expression on Day 3 of DSS feed. FIG. 10B is an image depicting a maximum projection of MPO expression. FIG. 10C is an image depicting a maximum projection of TNFα expression. FIG. 10 D is an image of a brightfield photomicrograph of a section stained with hematoxylin and eosin taken on Day 3.

FIG. 11, comprising FIG. 11A through FIG. 11H, is a series of images depicting maximum projection fluorescent images of tissue sections from animals taken on Day 6 of DSS feed. FIG. 11A is an image depicting a maximum projection depicting of IL1α expression. FIG. 11B is am image depicting a maximum projection depicting MPO expression. FIG. 11C is an image depicting a maximum projection of TNFα expression. FIG. 11D is an image of a brightfield photomicrograph of a section stained with hematoxylin and eosin. FIG. 11E is an image depicting a maximum projection depicting of IL1α expression. FIG. 11F is an image depicting a maximum projection depicting MPO expression. FIG. 11G is an image depicting a maximum projection of TNFα expression. FIG. 11H is an image of a brightfield photomicrograph of a section stained with hematoxylin and eosin.

FIG. 12, comprising FIG. 12A through FIG. 12 I, is a series of images depicting maximum projection images for 3 biomarkers from Day 7 of DSS feed. FIG. 12A is an image depicting IL1α expression labeled with 605QDs. FIG. 12B is an image depicting MPO expression. FIG. 12C is an image depicting TNFα expression labeled with 705QDs. FIG. 12D is an image depicting co-localization of three biomarkers labeled with three different QDs. FIG. 12E is an image of a brightfield photomicrograph of a section stained with hematoxylin and eosin. FIG. 12F is an image depicting IL1α expression labeled with 605QDs. FIG. 12G is an image depicting MPO expression. FIG. 12H is an image depicting TNFα expression labeled with 705QDs. FIG. 12I is an image of a brightfield photomicrograph of a section stained with hematoxylin and eosin.

FIG. 13, comprising FIG. 13A through FIG. 13D, is a series of images depicting maximum projection images for 3 biomarkers from Day 14 of the chronic stage of inflammation in the DSS model of UC. FIG. 13A is an image of a maximum projection image depicting IL1α expression. FIG. 13B is an image depicting a maximum projection image depicting MPO expression. FIG. 13C is an image of a maximum projection image depicting TNFα expression. FIG. 13D is an image of a brightfield photomicrograph of a section stained with hematoxylin and eosin.

FIG. 14, comprising FIG. 14A through FIG. 14D, is a series of images depicting maximum projection images for 3 biomarkers from Day 21 of the chronic stage of inflammation in the DSS model of UC. FIG. 14A is an image of a maximum projection image depicting IL1α expression. FIG. 14B is an image depicting a maximum projection image depicting MPO expression. FIG. 14C is an image of a maximum projection image depicting TNFα expression. FIG. 14D is an image of a brightfield photomicrograph of a section stained with hematoxylin and eosin.

### DETAILED DESCRIPTION OF THE INVENTION

The present invention includes a method of detecting one or more biomarkers to identify individuals with inflammatory disease using Quantum Dots conjugated to targeting moieties that specifically bind to a biomarker protein or a nucleic acid encoding a biomarker, where dysregulation of the biomarker is associated with inflammatory disease.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The phrase “biological sample” as used herein, is intended to mean any sample comprising a cell, a tissue, or a bodily fluid obtained from an organism in which expression of a biomarker can be detected. An example of such a biological sample includes a “body sample” obtained from a human patient. A “body sample” includes, but is not limited to blood, lymph, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

The term “dysregulation” as used herein is used describes an over- or under-expression of a biomarker present and detected in a biological sample obtained from a putative at-risk individual, then compared with a biomarker in a sample obtained from one or more normal, not-at-risk individuals. In some instances, the level of biomarker expression is compared with an average value obtained from more than one not-at-risk individuals. In other instances, the level of biomarker expression is compared with a biomarker level assessed in a sample obtained from one normal, not-at-risk sample. In yet another instance, the level of biomarker expression in the putative at-risk individual is compared with the level of biomarker expression in a sample obtained from the same individual at a different time.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “quantum dot” as used herein, is a semiconductor nanostructure that confines the motion of conduction band electrons, valence band holes, or excitons (bound pairs of conduction band electrons and valence band holes) in all three spatial directions. The confinement can be due to electrostatic potentials (generated by external electrodes, doping, strain, impurities), the presence of an interface between different semiconductor materials (e.g. in core-shell nanocrystal systems), the presence of the semiconductor surface (e.g. semiconductor nanocrystal), or a combination of these. A quantum dot (QD) has a discrete quantized energy spectrum. The corresponding wave functions are spatially localized within the quantum dot, but extend over many periods of the crystal lattice. A quantum dot contains a small finite number (of the order of 1-100) of conduction band electrons, valence band holes, or excitons, i.e., a finite number of elementary electric charges. One of the optical features of small excitonic quantum dots immediately noticeable to the unaided eye is coloration. While the material which makes up a quantum dot defines its intrinsic energy signature, more significant in terms of coloration is the size. The larger the dot, the redder (the more towards the red end of the spectrum) the fluorescence. The smaller the dot, the bluer (the more towards the blue end) it is. The coloration is directly related to the energy levels of the quantum dot. Quantitatively speaking, the bandgap energy that determines the energy (and hence color) of the fluoresced light is inversely proportional to the square of the size of the quantum dot.

As used herein, “conjugated” refers to a physical or chemical attachment of one molecule to a second molecule.

By the term “specifically binds,” as used herein, is meant a molecule, such as an antibody, which recognizes and binds to a cell surface molecule or feature, but does not substantially recognize or bind other molecules or features in a sample.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

“Inflammatory condition,” as the term is used herein, refers generally to a continued presence of inflammation in a mammal past the initial, beneficial immune response. Inflammatory conditions include, but are not limited to, chronic wounds, arthritis, atherosclerosis, and inflammatory diseases, such as autoimmune diseases, stroke, cardiovascular disease, acute coronary syndromes, acute myocardial infarction, pericarditis, periodontal disease, cancer in terms of it's connection to inflammatory disease, Alzheimer's disease, and inflammatory bowel disease.

### DESCRIPTION

Inflammatory disease is a complex, multifactorial sequelae characterized by severe derangements in the structure and function of local tissue architecture and increased presence of neutrophils and lymphocytes and other pro-inflammatory cells. In addition, epithelial, endothelial, mesenchymal, adipose tissue and nerve cells all can exhibit a broad range of damage as a result of the inflammatory process. Effector, regulatory and immune-like functions interact abnormally with lymphoid cells to further contribute to the pathogenesis of inflammatory disease. Heart disease, arthritis, asthma, allergy, infection and diabetes all have elements of chronic inflammation. Examples of inflammatory disease also include, but are not limited to, stroke, cardiovascular disease, acute coronary syndromes, acute myocardial infarction, pericarditis, periodontal disease, cancer, Alzheimer's disease, and inflammatory bowel disease. Inflammatory disease can also affect multiple organ systems, as in autoimmune diseases.

Inflammation is a significant contributor to the pathogenesis of both the acute and chronic stages of inflammatory bowel disease (IBD). One of the most common forms of IBD, Ulcerative Colitis, carries a significant risk for the development of colorectal cancer, but remains difficult to differentiate from another common form of IBD, Crohn's Disease.

### Biomarkers

A “biomarker” is any gene, protein, or metabolite whose level of expression in a tissue, cell or bodily fluid is dysregulated compared to that of a normal or healthy cell, tissue, or biological fluid. In one embodiment, a biomarker to be measured according to the method of the invention selectively responds to the presence and progression of inflammatory disease in an individual. By “selectively respond to the presence and progression of inflammatory disease” it is intended that the biomarker of interest is specifically over- or under-expressed in response to the onset and subsequent progression of inflammatory disease in an individual. This biomarker is not dysregulated during the course of other diseases, or other conditions not considered to be clinical disease. Thus, measuring the levels of biomarkers in the methods of the invention permits differentiation between samples collected from an individual with inflammatory disease and an individual without inflammatory disease.

A biomarker that can be measured according to the invention includes proteins and variants and fragments thereof, that exhibit dysregulation during inflammatory disease. Biomarker nucleic acids useful in the invention should be considered to include both DNA and RNA comprising the entire or partial sequence of any of the nucleic acid sequences encoding the biomarker, or the complement of such a sequence. Similarly, a biomarker protein should be considered to comprise the entire or partial amino acid sequence of any of the biomarker proteins or polypeptides.

In another embodiment of the invention, a biomarker to be measured selectively responds to the onset and progression of inflammatory bowel disease. By “selectively respond to the presence and progression of inflammatory bowel disease” it is intended that the biomarker of interest is specifically over- or under-expressed in response to the onset and subsequent progression of inflammatory bowel disease in an individual. This biomarker is not dysregulated during the course of other diseases of the bowel, or other conditions not considered to be clinical disease. Thus, measuring the levels of biomarkers in the methods of the invention permits differentiation between samples collected from an individual with inflammatory bowel disease and an individual without inflammatory bowel disease. In one aspect of the invention, the inflammatory bowel disease is ulcerative colitis. In another aspect of the invention, the inflammatory bowel disease is Crohn's Disease. Further, by measuring the levels of the biomarkers in the method of the invention, a practitioner would be able to distinguish different forms of IBD, specifically UC from CD.

By way of a non limiting example, serological samples obtained from patients with IBD that are positive for perinuclear antineutrophil cytoplasmic antibody (pANCA) but negative for anti-Saccharomyces cerevisiae antibody (ASCA) are indicative of ulcerative colitis, while serological samples positive for ASCA but negative for pANCA are indicative of Crohn's disease (Beaven and Abreu, 2004, Curr. Opin. Gastroent. 20:318-327). Biomarkers useful in the present invention include myeloperoxidase (MPO), IL1α and TNFα. Other biomarkers useful in the present invention include, but are not limited to, perinuclear anti-neutrophil cytoplasmic antibody (p-ANCA, Beaven and Abreu, 2004, Curr. Opin. Gastroent. 20:318-327); anti-Saccharomyces cerevisiae antibody (ASCA, Beaven and Abreu, 2004, Curr. Opin. Gastroent. 20:318-327); angiotensin converting enzyme (Kwon et al., 2007, Korean J. Intern. Med. 22:1-7); lactoferrin (Walker et al., 2007, J. Pediatric Gastroent. Nutr. 44:414-422); C-reactive protein (CRP, von Roon et al., 2007, Am. J. Gastroenterol. 102:803-813); and calprotectin (von Roon et al., 2007, Am. J. Gastroenterol. 102:803-813).

In another embodiment, the present invention provides for analogs of polypeptides which comprise a biomarker protein. Analogs may differ from naturally occurring proteins or polypeptides by conservative amino acid sequence differences or by modifications which do not affect sequence, or by both. For example, conservative amino acid changes may be made, which although they alter the primary sequence of the protein or polypeptide, do not normally alter its function (e.g., secretion and capable of blocking virus infection). Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

The present invention should also be construed to encompass “mutants,” “derivatives,” and “variants” of the biomarker proteins of the invention (or of the DNA encoding the same) which mutants, derivatives and variants are altered in one or more amino acids (or, when referring to the nucleotide sequence encoding the same, are altered in one or more base pairs) such that the resulting peptide (or DNA) is not identical to the sequences recited herein, but has the same biological property as the biomarker proteins disclosed herein, in that the proteins have biological/biochemical properties. A biological property of the polypeptides of the present invention should be construed but not be limited to include, their regulation during inflammation.

Further, the invention should be construed to include naturally occurring variants or recombinantly derived mutants of biomarker protein sequences, which variants or mutants render the polypeptide encoded thereby either more, less, or just as biologically active as wild type biomarker protein.

In one embodiment, the biological activity of a biomarker of the invention is the ability of the biomarker to respond in a predictable way to the onset and progression of inflammatory disease. In another embodiment of the invention, the biological activity of the biomarker is to respond in a predictable way to the onset and progression of inflammatory bowel disease. In one aspect, a biomarker responds to the onset and progression of ulcerative colitis. In another aspect, a biomarker responds to the onset and progression of Crohn's Disease.

Biomarkers of the invention include, but are not limited to, an enzyme, an adhesion molecule, a cytokine, a protein, a lipid mediator, and a growth factor. In an embodiment, biomarkers of the invention include, but are not limited to, myeloperoxidase (MPO), IL1α and TNFα, perinuclear anti-neutrophil cytoplasmic antibody (p-ANCA, Beaven and Abreu, 2004, Curr. Opin. Gastroent. 20:318-327); anti-Saccharomyces cerevisiae antibody (ASCA, Beaven and Abreu, 2004, Curr. Opin. Gastroent. 20:318-327); angiotensin converting enzyme (Kwon et al., 2007, Korean J. Intern. Med. 22:1-7); lactoferrin (Walker et al., 2007, J. Pediatric Gastroent. Nutr. 44:414-422); C-reactive protein (CRP, von Roon et al., 2007, Am. J. Gastroenterol. 102:803-813); and calprotectin (von Roon et al., 2007, Am. J. Gastroenterol. 102:803-813).

Although a method of the invention requires the detection of at least one biomarker in a body sample, two or more biomarkers may be used to practice the method of the present invention. Therefore, in an embodiment, two or more biomarkers are used. In an aspect of the invention, two or more complementary biomarkers are used.

When used to refer to a biomarker herein, the term “complementary” is intended to mean that detection of the combination of biomarkers in a body sample results in the successful identification of a patient with inflammatory disease in a greater percentage of cases than would be identified if only one biomarker was used. In one embodiment of the invention, two biomarkers may be used to more accurately identify a patient with IBD than when one biomarker is used. In one aspect of the invention, two or more biomarkers may be used to diagnose ulcerative colitis. In another aspect of the invention, two or more biomarkers are used to identify a patient with Crohn's Disease.

Accordingly, where at least two biomarkers are used, at least two antibodies directed to distinct biomarker proteins will be used to practice the immunocytochemistry methods disclosed herein. The antibodies may be contacted with the body sample simultaneously or sequentially.

### Reporter Components

A conjugate of the present invention encompasses at least one reporter component. In one embodiment, a reporter component of the invention includes, but is not limited to a quantum dot, wherein said quantum dot is detected by means of its fluorescent properties. In another embodiment, a magnetic nanoparticle can be used in the same manner as described for fluorescent QD except that detection of magnetic nanoparticle would be achieved using means including but not limited to a SQUID (Superconducting Quantum Interference Device), fluxgate magnetometer or other device used in the art to detect the presence of magnetic moments of small magnetic fields (Schwartz et al., 2003, J. Am. Chem. 125:13205-13218). In yet another embodiment, a reporter component comprises a magnetic quantum dot with both fluorescent and magnetic properties.

Therefore, in one embodiment, the present invention encompasses semiconductor nanocrystals, also known as Quantum Dots (QD), as ultra-sensitive non-isotopic reporters of biomolecules in vitro and in vivo. QDs are attractive fluorescent tags for biological molecules due to their large quantum yield and photostability. As such, QD overcome many of the limitations inherent to the organic dyes used as conventional fluorophores. QD range from 2 nm to 10 nm in diameter, contain approximately 500-1000 atoms of materials such as cadmium and selenide, and fluoresce with a broad absorption spectrum and a narrow emission spectrum.

A water-soluble luminescent QD, which comprises a core, a cap and a hydrophilic attachment group is well known in the art and commercially available (e.g. Quantum Dot Corp. Hayward, Calif.; Invitrogen, Carlsbad, Calif.; U.S. Pat. No. 7,192,785; U.S. Pat. No. 6,815,064). The “core” comprises a nanoparticle-sized semiconductor. While any core of the IIB VIB, IIIB VB or IVB-IVB semiconductors can be used, the core must be such that, upon combination with a cap, a luminescence results.

The “cap” is a semiconductor that differs from the semiconductor of the core and binds to the core, thereby forming a surface layer on the core. The cap must be such that, upon combination with a given semiconductor core, a luminescence results. Two of the most widely used commercial QDs come with a core of CdSe or CdTe with a shell of ZnS and emissions from 405 nm to 805 nm.

The “attachment group” as used herein, refers to any organic group that can be attached, such as by any stable physical or chemical association, to the surface of the cap of the QD. In one embodiment, the attachment group can render the QD water-soluble without rendering the QD no longer luminescent. Accordingly, the attachment group comprises a hydrophilic moiety. In one aspect, the attachment group may be attached to the cap by covalent bonding and is attached to the cap in such a manner that the hydrophilic moiety is exposed. Suitable hydrophilic attachment groups include, for example, a carboxylic acid or salt thereof, a sulfonic acid or salt thereof, a sulfamic acid or salt thereof, an amino substituent, a quaternary ammonium salt, and a hydroxy. In another aspect, QD may be rendered water soluble by capping the shell with a polymer layer that contains a hydrophobic segment facing inside towards the shell and a hydrophilic segment facing outside. The hydrophilic layer can be modified to include functional groups such as —COOH and —NH₂ groups for further conjugation to proteins and antibodies or oligonulceotides as described in Chan and Nie, 1998, (Science 281:2016-8), Igor et al., 2005, (Nature Materials 4:435-46), Alivisatos et al., 2005, (Annu. Rev. Biomed. Eng. 7:55-76) and Jaiswal et al., 2003, (Nature Biotech. 21:47-51) and incorporated herein in their entirety by reference.

### QD Conjugates

The present invention also provides a conjugate comprising a water-soluble QD, as described above, conjugated to a targeting moiety. The targeting moiety specifically binds to the biomarker of interest and may comprise an antibody, a peptidomimetic, a polypeptide or aptamer, a nucleic acid or any other molecule provided it binds specifically to a biomarker of interest.

In one embodiment, the QD may be conjugated to a targeting moiety comprising an antibody. Preferably, the antibody specifically binds to a biomarker that is dysregulated during the onset and progression of inflammatory disease. In another embodiment, the antibody specifically binds to a biomarker that is dysregulated by the onset and progression of inflammatory bowel disease. In another embodiment, the antibody specifically binds to a biomarker that is dysregulated by the onset and progression of ulcerative colitis. In still another embodiment, the antibody specifically binds to a biomarker that is dysregulated during the onset and progression of Crohn's Disease. Biomarkers of interest in the present invention include, but are not limited to, MPO, or cytokines involved in inflammation, such as IL1α or TNFα.

In another embodiment, the QD may be conjugated to a targeting moiety comprising a nucleic acid binding moiety. The nucleic acid binding moiety may comprise any nucleic acid, protein, or peptide that binds to nucleic acids, such as a DNA binding protein. A preferred nucleic acid is a single-stranded oligonucleotide comprising a stem and loop structure and the hydrophilic attachment group is attached to one end of the single-stranded oligonucleotide.

The antibody or nucleic acid can be attached to the QD, such as by any stable physical or chemical association, directly or indirectly by any suitable means. Quantum Dot (QD) conjugation may be achieved by a variety of strategies that include but are not limited to passive adsorption, multivalent chelates or classic covalent bond formation described in Jaiswal et al., 2003 (Nature Biotechnol. 21:47-51) and incorporated by reference herein.

The covalent bond formation is the simplest in execution and hence widely used for conjugation. The antibody or nucleic acid is attached to the attachment group directly or indirectly through one or more covalent bonds. If the antibody is attached indirectly, the attachment preferably is by means of a “linker.” Use of the term “linker” is intended to encompass any suitable means that can be used to link the antibody or nucleic acid to the attachment group of the water-soluble QD. The linker should not render the water-soluble QD water-insoluble and should not adversely affect the luminescence of the QD. Also, the linker should not adversely affect the function of the attached antibody or nucleic acid. If the conjugate is to be used in vivo, desirably the linker is biologically compatible. Crosslinkers, e.g. intermediate crosslinkers, can be used to attach an antibody to the attachment group of the QD. Ethyl-3-(dimethylaminopropyl) carbodiimide (EDAC) is an example of an intermediate crosslinker. Other examples of intermediate crosslinkers for use in the present invention are known in the art. See, for example, Bioconjugate Techniques (Academic Press, New York, (1996)).

In one embodiment, amine groups on QDs are treated with a malemide group containing a crosslinker molecule. These “activated” QDs can be then be directly conjugated to a whole antibody molecule. However the direct conjugation may result in steric hindrance restricting access of the antibody to the antigen of interest. In those instances where a short linker could cause steric hindrance problems or otherwise affect the functioning of the targeting moiety, the length of the linker can be increased, e.g., by the addition of from about a 10 to about a 20 atom spacer, using procedures well-known in the art (see, for example, Bioconjugate Techniques (1996), supra). One possible linker is activated polyethylene glycol, which is hydrophilic and is widely used in preparing labeled oligonucleotides.

The Stretptavidin Biotin reaction provides another conjugation method where the biotinylated protein/biomolecule is attached to a streptavidin coated QD.

### Antibodies

When the antibody conjugated to the QD is a polyclonal antibody (IgG), the antibody is generated by inoculating a suitable animal with the targeted cell surface molecule. Antibodies produced in the inoculated animal which specifically bind to the cell surface molecule are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1988, In: Antibodies, A Laboratory Manual, Cold Spring Harbor, N.Y.).

Monoclonal antibodies directed against a full length targeted cell surface molecule or fragments thereof may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein.

When the antibody used in the methods of the invention is a biologically active antibody fragment or a synthetic antibody corresponding to antibody to a targeted cell surface molecule, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with targeted cell surface molecule epitopes. These antibodies are capable of binding to the targeted cell surface molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Hematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the targeted cell surface molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1988, Antibodies: A Laboratory Manual, Cold Spring Harbor, N.Y.).

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Samples may need to be modified in order to render the target molecule antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO 51699)/dH₂O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a biomarker of interest is then incubated with the sample.

One of skill in the art will appreciate that it may be desirable to detect more than one protein of interest in a biological sample. Therefore, in particular embodiments, at least two antibodies directed to two distinct proteins are used. Where more than one antibody is used, these antibodies may be added to a single sample sequentially as individual antibody reagents or simultaneously as an antibody cocktail. Alternatively, each individual antibody may be added to a separate sample from the same source, and the resulting data pooled.

### Detection Using QD as Fluorophores

Given the disclosure set forth herein, the skilled artisan will understand how to use any methods available in the art for identification or detection of a protein, nucleic acid, or a biomolecule of interest. Methods for detecting a molecule of interest comprise any method that determines the quantity or the presence of the biomarker protein or nucleic acid.

The invention should not be limited to any one method of protein, nucleic acid, or biomolecule detection method recited herein, but rather should encompass all known or heretofore unknown methods of detection as are, or become, known in the art.

In one embodiment, the biomarker of interest is detected at the protein level. The method comprises contacting the sample with a QD-antibody conjugate as described above, wherein the antibody of the conjugate specifically binds to the biomarker protein and detecting fluorescence, wherein the detection of fluorescence indicates that the conjugate bound to a protein in the sample.

In one aspect, the method of the invention is used to detect a protein of interest in a biological sample using methods well known in the art that include, but are not limited to, western blots, ELISA, immunoprecipitation, immunofluorescence, flow cytometry, immunocytochemistry techniques.

In another embodiment, the target molecule of interest is detected at the nucleic acid level. The method comprises contacting the sample with a QD-conjugate as described above, wherein the targeting moiety of the conjugate specifically binds to the nucleic acid and detecting residual fluorescence, wherein the detection of fluorescence indicates that the conjugate bound to the nucleic acid in the sample. Preferably, the targeting moiety of the conjugate is a nucleic acid. Alternatively, the targeting moiety of the conjugate is a protein or a fragment thereof that binds to a nucleic acid, such as a DNA binding protein.

Nucleic acid-based techniques for assessing expression are well known in the art and include, for example, Northern and Southern blots, gene chip or microarrays, (Schena et al., 1995, Science 270:467-70; Gibson, 2003, PLoS Biol 1:e15), nucleic acid amplification, including detecting mRNA in a biological sample by RT-PCR. Many expression detection methods use isolated RNA. Any RNA isolation technique that does not select against the isolation of mRNA can be utilized for the purification of RNA from biological samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155).

The term “probe” refers to any molecule that is capable of selectively binding to a specifically intended target molecule, for example, a nucleotide transcript or a protein encoded by or corresponding to a target molecule. Probes can be synthesized by one of skill in the art, or derived from appropriate biological preparations. As contemplated in the present invention, a probe may be conjugated to an SCN of a particular size. Examples of molecules that can be used as probes include, but are not limited to, RNA, DNA, proteins, antibodies, and organic molecules.

The present invention also provides a method whereby two or more different target molecules and/or two or more regions on a given target molecule can be simultaneously detected in a sample. The method involves using a set of QD conjugates, wherein each of the conjugates in the set has a differently sized QD or a QD of different composition attached to a targeting moiety that specifically binds to a different target molecule or a different region on a given target molecule in the sample. In an embodiment, the QD of the conjugates range in size from 2 nm to 6.5 nm, which sizes allow the emission of luminescence in the range of blue to red. The QD size that corresponds to a particular color emission is well-known in the art. Within this size range, any size variation of QD can be used as long as the differently sized QD can be excited at a single wavelength and differences in the luminescence between the differently sized QD can be detected. In another embodiment, the differently sized QD have a capping layer that has a narrow and symmetric emission peak. Similarly, QD of different composition or configuration will vary with respect to particular color emission. Any variation of composition between QD can be used as long as the QD differing in composition can be excited at a single wavelength and differences in the luminescence between the QD of different composition can be detected. Detection of the different biomarkers in the sample arises from the emission of multicolored luminescence generated by the QD differing in composition or the differently sized QD of which the set of conjugates is comprised. This method also enables different functional domains of a single protein, for example, to be distinguished.

Accordingly, the present invention provides a method of simultaneously detecting two or more different biomarkers and/or two or more regions of a given biomarker in a sample. The method comprises contacting the sample with two or more conjugates of a water-soluble QD and an antibody, wherein each of the two or more conjugates comprises a QD of a different size or composition and an antibody that specifically binds to a different molecule or a different region of a given target molecule in the sample. The method further comprises detecting luminescence, wherein the detection of luminescence of a given color is indicative of a conjugate binding to a molecule in the sample.

The above described conjugates and methods can be adapted for use in numerous other methods and biological systems to effect the detection of a biomarker. Such methods are well known in the art and include but are not limited to western blots, ELISA, immunoprecipitation, immunofluorescence, flow cytometry, and immunocytochemistry methods.

### Diagnostic Assays

The present invention has application in various diagnostic assays, including, but not limited to, the detection of any inflammatory disease, including, but not limited to IBD, UC, and CD. The present invention can be used to detect inflammatory disease such as IBD by removing a sample to be tested from a patient; contacting the sample with a water-soluble QD conjugated to a targeting moiety that specifically binds to a biomarker associated with a given disease state and detecting the luminescence, wherein the detection of luminescence indicates the existence of a given disease state, such as IBD. In these cases, the sample can be a cell or tissue biopsy or a bodily fluid, such as blood, serum, urine, or fecal sample.

The biomarker can be a protein, a nucleic acid or enzyme associated with a given disease, the detection of which indicates the existence of a given disease state. The detection of a disease state can be either quantitative, as in the detection of an over- or under-production of a protein, or qualitative, as in the detection of a non-wild-type (mutated or truncated) form of the protein. In regard to quantitative measurements, preferably the luminescence of the QD conjugate is compared to a suitable set of standards. A suitable set of standards comprises, for example, the QD conjugate of the present invention in contact with various, predetermined concentrations of the biomarker being detected. One of ordinary skill in the art will appreciate that an estimate of, for example, amount of protein in a sample, can be determined by comparison of the luminescence of the sample and the luminescence of the appropriate standards, as described in detail elsewhere herein.

The above-described methods also can be adapted for in vivo testing in an animal. In one embodiment, the conjugate is administered to the animal in a biologically acceptable carrier. The route of administration should be one that achieves contact between the conjugate and the targeting moiety, e.g., protein or nucleic acid, to be assayed. The in vivo applications are limited only by the means of detecting the biomarker-QD conjugate. In other words, the site of contact between the conjugate and the biomolecule to be assayed must be accessible by a optical detection means. In this regard, fiber optics can be used. An optical fiber is an optical waveguide and acts as a conduit of optical signal by confining light to the fiber core due to total internal reflection at the fiber core/cladding interface. A suitably designed optical fiber probe can transport optical signal to and from the region of interest as needed in the context of present invention.

### Kits

Kits for practicing the methods of the invention are further provided. By “kit” is intended any manufacture (e.g., a package or a container) comprising at least one reagent, e.g., an antibody, a nucleic acid probe, etc. for specifically detecting the expression of a biomarker of the invention. The kit may be promoted, distributed, or sold as a unit for performing the methods of the present invention. Additionally, the kits may contain a package insert describing the kit and including instructional material for its use.

In a particular embodiment, kits for practicing the immunocytochemistry methods of the invention are provided. Such kits are compatible with both manual and automated immunocytochemistry techniques (e.g., cell staining). These kits comprise at least one antibody directed to a biomarker of interest, chemicals for the detection of antibody binding to the biomarker, a counterstain, and, optionally, a counterstain to facilitate identification of positive staining cells. Other reagents may be further provided in the kit to facilitate detection of positive staining cells.

In another embodiment, the immunocytochemistry kits of the invention additionally comprise at least two reagents, e.g., antibodies, for specifically detecting the expression of at least two distinct biomarkers. Each antibody may be provided in the kit as an individual reagent or, alternatively, as an antibody cocktail comprising all of the antibodies directed to the different biomarkers of interest. Furthermore, any or all of the kit reagents may be provided within containers that protect them from the external environment, such as in sealed containers.

Positive and/or negative controls may be included in the kits to validate the activity and correct usage of reagents employed in accordance with the invention. Controls may include samples, such as tissue sections, cells fixed on glass slides, etc., known to be either positive or negative for the presence of the biomarker of interest. The design and use of controls is standard and well within the routine capabilities of those of ordinary skill in the art.

One of skill in the art will further appreciate that any or all steps in the methods of the invention could be implemented by personnel or, alternatively, performed in an automated fashion. Thus, the steps of body sample preparation, sample staining, and detection of biomarker expression may be automated.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Quantum Dot Conjugation

Quantum Dots were conjugated to antibody fragments using a heterobiofunctional crosslinker 4-(maleimidomethyl)-1-cyclohexanecarboxylic acid N-hydroxysuccinimide ester (SMCC). The commercial Quantum Dots (QDs) (Invitrogen Corporation, Carlsbad, Calif.) come with —NH₂ groups on their surface. These amino groups are reacted with the crosslinker SMCC to create malemide groups on the QDs surface. Antibodies of interest are reduced by DTT (Dithiothreitol) and disulfide bonds are broken to create thiol (—SH) groups. The final conjugation relied on the covalent bond formed between the malemide group on activated QDs and the thiol group on the antibodies. The ratio of antibody conjugated to QDs is 1:4 and the typical yield of the reaction at the end of conjugation procedure is anywhere between 500 μl to 800 μl. Table I presents a list of QDs conjugated to antibodies using the procedure outlined above:

TABLE 1
Different color QDs conjugated to various antibodies.

| | | |
|---|---|---|
| Quantum Dots | Antibodies | Stock Concentration |

| | | | |
|---|---|---|---|
| QD565 | MPO (Santa Cruz BT) | 1.2 | μM |
| QD655 | MPO (Santa Cruz BT) | 500 | nM |
| QD655 | Anti- Testosterone | 1.5 | μM |
| QD605 | Anti-TNFα | 1 | μM |
| QD705 | Anti-TNFα | 1.2 | μM |
| QD 605 | Anti-IL1α | 1.5 | μM |
| QD 705 | Anti-IL1α | 1.5 | μM |

### Dextran Sodium Sulfate (DSS) Model of Colitis

The Dextran Sodium Sulfate (DSS) model of ulcerative colitis (UC) is a well established animal model of human ulcerative colitis. The DSS model is an attractive model of human disease because of the simplicity of disease induction, reproducible time course of disease development, and relative uniformity of lesions (Cooper et al., 1993, Lab Invest. 69:238-49; Murthy and Flangian, 1999, Animal models of IBD. In vivo model of inflammation, ed. Morgan and Marshall, Birkhause publication Switzerland, 210-32). Hence this model has been extensively used for studying the pathology behind ulcerative colitis and the development of various therapeutic regimes like anticytokine therapy, anticancer therapy, antiadhesion molecules, nitric oxide inhibitors, metabolite synthesis and receptors and arachidionic acid inhibitors (Murthy and Flangian, 1999, Animal models of IBD. In vivo model of inflammation, ed. Morgan and Marshall, Birkhause publication Switzerland, 210-32; Kojouharoff et al., 1997, Clin. Exp. Immunol. 107:353-8; Yoshinori et al., 1998, Cytokine 9:789-95; Leo et al., 2000, Digest. Dis. And Sci. 45:2327-2336; Jun-Ichi et al., 2002, J. Gastroent. And Hepatol. 17:1291-8).

The disease is induced in mice and rats by oral administration of Dextran Sodium Sulfate dissolved in water. Animals develop acute inflammation by the 3rd day after the start of the DSS cycle and by the 7th day the animals have severe acute inflammation corresponding to the clinical presentation of full-blown ulcerative colitis (Nida et al, 2005, Gynecol. Oncol. 99:S89-94; Cooper et al., 1993, Lab Invest. 69:238-49). Chronic inflammation is developed after animals are revert back to plain water feeding for 14 days from the stop of DSS feed (Nida et al, 2005, Gynecol. Oncol. 99:S89-94; Cooper et al., 1993, Lab Invest. 69:238-49).

Acute inflammation is marked by infiltration of the mucosal layer with an increasing number of neutrophils, progressive shortening of epithelial crypts, hyalination in the lamina propria and severe weight loss marked with bloody stools and diarrhea (Cooper et al., 1993, Lab Invest. 69:238-49; Murthy and Flangian, 1999, Animal models of IBD. In vivo model of inflammation, ed. Morgan and Marshall, Birkhause publication Switzerland, 210-32). The model resembles human ulcerative colitis by many criteria including increased reactive oxygen metabolites (ROM) levels in the mucosa, superficial ulceration, increased production of cytokines and other inflammatory mediators as well as increased leukocyte infiltration of lamina propria.

Chronic inflammation in the DSS model is marked by a regenerating crypt layer and infiltration of mucosal layer with a mixed infiltrate of macrophages, monocytes, and lymphocytes with a minor component of neutrophils. Induction of chronic inflammation in the DSS model permits the evaluation of therapeutic compounds' efficacy (Murthy and Flangian, 1999, Animal models of IBD. In vivo model of inflammation, ed. Morgan and Marshall, Birkhause publication Switzerland, 210-32).

Though the exact mechanism of disease induction and pathogenesis is unknown, it is believed that DSS most likely overcomes the epithelial barrier exposing the mucosal layer to colonic microflora and resulting in an inflammatory response (Vowinkel et al., 2004, Digest. Disease. And Sci. 49:556-564; Katajima et al., 1999, Exp. Animal 48:137-143). DSS is also suspected to activate macrophages and monocytes (Cooper et al., 1993, Lab Invest. 69:238-49; Katajima et al., 1999, Exp. Animal 48:137-143).

Disease progression is measured by a functional clinical symptom composite score along with a histology score. The functional score is based on subject weight loss, hemocult tests for blood in the stools, and stool consistency. The functional score exhibits excellent correlation with the histology score based on the crypt architectural changes (Cooper et al., 1993, Lab Invest. 69:238-49).

Female Swiss Webster mice of approximately 8 weeks of age (25 to 30 gms in weight) were housed in a separate cage. Inflammation was induced by feeding ad libitum 4% DSS, molecular weight approximately 40,000 (ICN, Costa Mesa, Calif.) in their drinking water. For chronic inflammation, after 7 days of the DSS feeding cycle, mice were put back on normal tap water for a period of 21 days. The animals were continuously monitored for weight loss, stool consistency and blood in stools during the whole length of studies.

An accepted measure of disease progression in this animal model of inflammation is a composite index of clinical parameters, referred to as the Disease Activity Index (DAI) (Cooper et al., 1993, Lab Invest. 69:238-49; Murthy and Flangian, 1999, Animal models of IBD. In vivo model of inflammation, ed. Morgan and Marshall, Birkhause publication Switzerland, 210-32). The DAI was determined in all animals, by scoring body weight, hemocult reactivity or presence of gross blood in stools and stool consistency, as detailed in previous studies (Cooper et al., 1993, Lab Invest. 69:238-49; Murthy and Flangian, 1999, Animal models of IBD. In vivo model of inflammation, ed. Morgan and Marshall, Birkhause publication Switzerland, 210-32). A scale of 0-3 is used to score the DAI, 3 representing the most severe clinical disease and 0 the least severe clinical disease. DAI was measured starting from Day 3 daily, until the score reached 3 which generally occur on Day 7 or Day 8 of the DSS feeding cycle. For chronic inflammation the procedure was repeated for all the days.

### Surgical Preparation

Mice were anesthetized by an i.p. dose of sodium pentobarbital (Abbott Laboratories, Illinois). Animal body temperature was maintained using an overhead lamp. A laparatomy was performed and the colon exposed. The colon was exteriorized and two catheters inserted, creating a loop involving the distal colon. One of the catheters, referred as infusion catheter (PE 60, 1 mm in outside diameter) was positioned into the proximal colon through the cecum and secured by suturing it to the colon. The second catheter, a drainage catheter (PE 60, 1 mm in outside diameter), was introduced through the rectum and positioned approximately 1 cm above the anal verge and secured by a surgical suture. The colon was thoroughly washed with saline.

Since in the DSS model of colitis the disease is generally limited to the distal colon (Cooper et al., 1993, Lab Invest. 69:238-49; Murthy and Flangian, 1999, Animal models of IBD. In vivo model of inflammation, ed. Morgan and Marshall, Birkhause publication Switzerland, 210-32), a volume of approximately 80 μl QD-conjugate was introduced through the drainage catheter. The QD conjugates were allowed to remain in contact with the mucosa for approximately fifteen minutes after which they were drained and the colon was washed thoroughly with 9 ml of saline and the distal part colon surgically removed, cut opened, again washed thoroughly with saline and then snap frozen. The animal euthanized with an overdose of pentobarbital. The frozen tissue is embedded in a water soluble specimen matrix (Tissue Tek O.C.T. Compound; Sakura Finetek US, Torrance, Calif.) and kept at −90° C. until sectioned.

For DSS fed animals, the procedure described above was performed on various days after DSS feeding had begun, starting from Day 3 until the DAI reached 3.00 or the animal was in severe distress.

### Sectioning and Confocal Microscopy

Thin sections of approximately 15 microns were cut with a cryostat and mounted on gelatin coated glass slides. Sections were fixed with Ethyl alcohol according to established protocols. Briefly, the frozen sections were kept at room temperature for 30 minutes. The sections were dipped in 100%, 90%, 70% and 50% Ethanol each for a period of 10 minutes. The sections were further washed in deionized water for a period of 15 minutes to remove the O.T.C. freezing medium matrix. The slides were dried, mounted with fluorescent medium with or without DAPI counterstain and covered with a glass coverslip for confocal microscopy. The mounted sections were imaged with a multiphoton Leica Sp2 confocal microscope (Leica Microsystems Inc., Bannockburn, Ill.). Images were acquired with a 40× objective with an Argon laser (excitation wavelength of 488 nm) as an excitation source. The emission wavelengths were optimized by calibrating the confocal microscope for all the QDs. For this purpose, an emission wavelength window was determined for each QDs separately and when mixed in different combinations. Optimized emission wavelength windows were obtained especially for 605, 655 and 705QDs since these QDs were used together for three different marker studies. Following are the emission wavelength (λ) window for the QDs used:

TABLE 2
Emission wavelength (λ) window for the QDs used.

| Type of QD | Emission λ window (nm) |
|---|---|
| 605 | 585-625 |
| 655 | 635-670 |
| 705 | 685-740 |

The Z-series image stacks (Z-stacks) were collected with a slice thickness of 0.7 microns along the z-axis. Transmitted light images were also collected in another channel. Photomultiplier gains for individual channels were optimized to achieve the optimal dynamic range for all the tissue sections images. Each optical sections (1 scan/image; 512×512 resolution) were collected for data analysis in a two dimensional optical mode.

Z-stacks were processed and maximum intensity projection images were obtained using the Leica image acquisition software. All subsequent image processing steps were carried on these projection images using Image J (National Institute of Mental Health, Bethesda, Md.) and Adobe Photoshop (Adobe Systems Incorporated, San Jose, Calif.).

### Hematoxylin and Eosin (H & E) Staining

Sections were stained for H&E to compare the localization of QDs with the H&E stained sections. Every 4 sections, one section was stained with H&E to have good histological localization of the QD stained tissue. H&E staining was performed according to established protocols. Briefly, frozen sections were kept at room temperature for 30 minutes. The sections were dipped in 100%, 90%, 70% and 50% Ethanol each for a period of 10 minutes. The sections were further washed in deionized water for a period of 15 minutes. The sections were then stained for Hematoxylin for 1 minute and rinsed in running tap water for 10 minutes. The slides were de-stained by dipping them quickly in 0.025% HCL in 70% Ethanol for a period of 20 seconds. The slides were again washed in running tap water for 10 minutes. The slides were then dipped in Eosin solution for 45 seconds and dipped in increasing concentration of Ethanol (50%, 70%, 90%, and 100%) for 10 minutes each. The slides were then dipped in Xyline and mounted with Permount®. The slides were imaged with Leica epifluorescent microscope using a 40× objective NA. No image processing was performed to the H&E images.

### Image Processing

All the confocal images were processed using Image J (Bethesda, Md.) (Abramoff et al., 2004, Biophotomics Intnl. 11:36-41). A minimum background was established for each using the ROI plug-in in Image J. Briefly, a region outside the tissue was selected and the pixels subtracted from the whole image giving a “clean image.” To obtain fluorescence intensity values of the QDs in the section, a multimeasure mode in the same ROI plugin was used. This was done for multiple image and the intensity values were exported to an excel file. The values were then plotted as a graph and compared with the functional clinical symptoms composite score (DAI) score that is representative of the experimental colitis disease. To correlate localization of QDs in the tissue to tissue morphology, QD projection images were superimposed on bright field images using AdobePhotoshop. Apart from the above mentioned processing no other image manipulation was carried out including no brightness or contrast manipulations.

The results of the experiments presented in this Example are now described.

### Example# 1

### DSS Induced Colitis

Colitis was induced as outlined in the materials and methods section and the induction was monitored by calculating Disease Activity Index (DAI). On Day 4 of the DSS feed animals developed colitis characterized by loose stool consistency. Blood in stools was typically detected on Day 4 in the hemoccult tests and was a consistent observation throughout the duration of the experiments. From Day 6 onwards most of the mice showed bloody diarrhea. Animals lost weight from Day 4 onward and had lost about 15% to 20% of their control weight by the end of Day 7.

The severity of colitis was assessed using an acceptable measure in the form of Disease Activity Index (DAI) (Cooper et al., 1993, Lab Invest. 69:238-49). The parameters employed in calculating DAI are based on the clinical symptoms observed in human ulcerative colitis and include weight loss, stool consistency (normal, loose, diarrhea) and presence or absence of blood in stools (hemoccult tests). The DAI scale ranges from 0 to 3 with 3 being the most severe form of the disease. This method of scoring is a comprehensive functional measure that correlated well with the degree of inflammation.

### Experiment # 2

### Imaging MPO Expression with QD565 in the DSS Model of Colitis

The DSS animal model of colitis is the most representative animal model for human ulcerative colitis. The model is characterized by the gradual induction of the disease until it is full blown. In this model neutrophil infiltration of the mucosal layer increases throughout the period of DSS feed and the crypt structure begins to noticeably deteriorate on Day 4 when the crypts start becoming shorter.

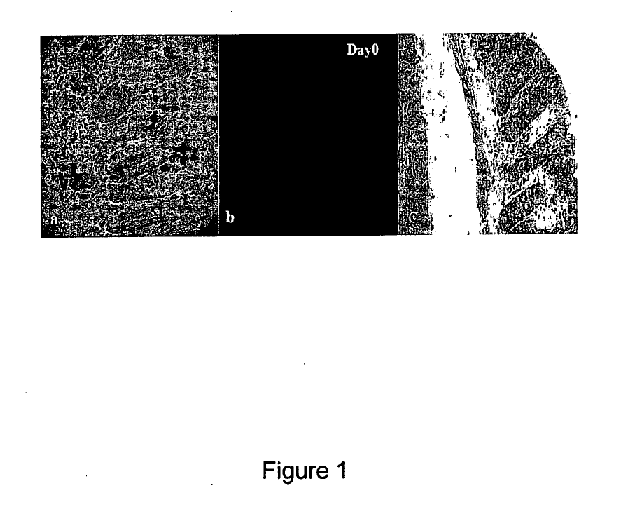

The experiment to target MPO in vivo with Quantum Dots used a 565QD-MPO antibody conjugate. Two DSS fed animals were examined per day of the experiment. The time course for each experiment ranged from Day 0 (FIG. 1; control) to Day 7.

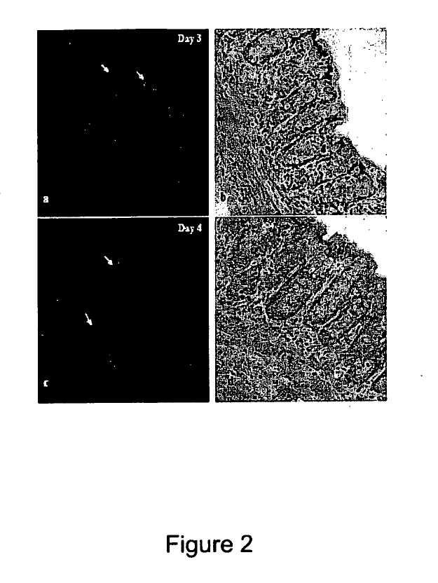

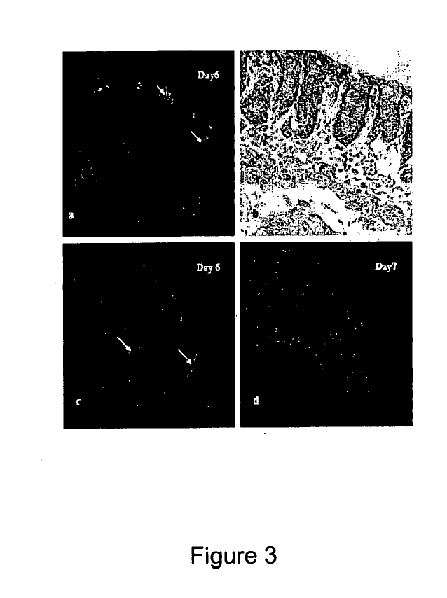

From the H&E stained images at different time points throughout the experiment it is clear that the crypts are becoming shorter and more neutrophils appear with increased inflammation. The fluorescent images are maximum projection images. The number of QDs and their intensity markedly increases from Day 3 (FIG. 2) to Day 7 (FIG. 3) suggesting a relationship with the inflammation.

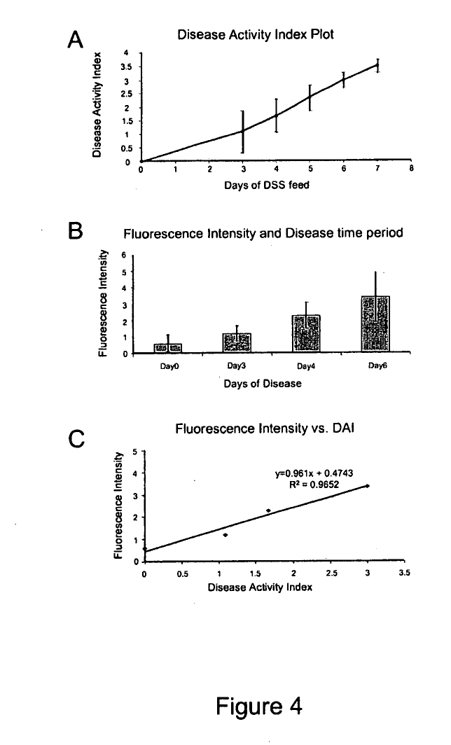

Though here the histology scores were not obtained, it is clear that the neutrophil count has increased from Day 3 to Day 6 of the DSS feed. FIG. 1, FIG. 2, and FIG. 3 were processed and intensity values were calculated using Image J software for all the days. The values were plotted against the DAI values obtained from the parameters monitored during the DSS feed cycle. FIG. 4 shows the DAI plot, fluorescence intensity plot against the days of DSS feed and fluorescence intensity versus DAI values plot.

It is clear that as the inflammation increases the expression level of MPO increases. However in this experiment the autofluorescence of tissues interfered with the fluorescence intensity and hence contributing some error in the data. Hence in the next set of experiments, QDs were red shifted to avoid issues with autofluorescence.

### Experiment # 3

### Imaging MPO Expression with QD655 in the DSS Model of Colitis

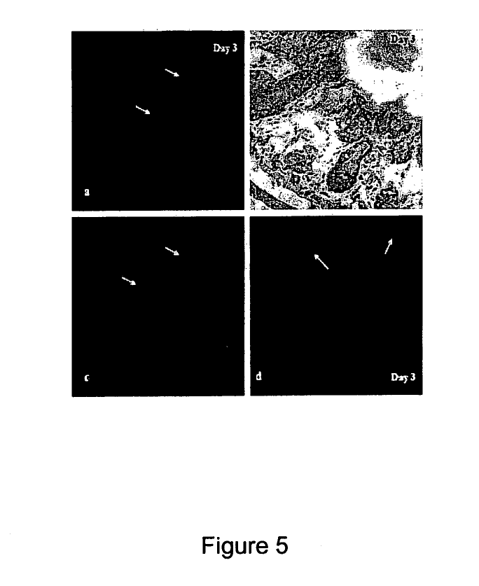

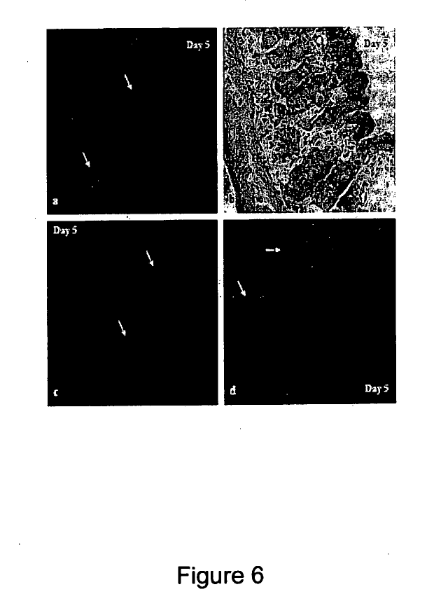

In this experiment MPO antibody were conjugated to red colored QDs with emission maximum at 655 nm. Six animals were used with two animals being used each on Day 3, Day 5 and Day 8 of the disease. Both H&E staining as well as confocal imaging were performed on all the sections obtained from the animals on respective days. On the Day 3 animals showed little loss in weight and the hemoccult tests didn't show any blood (FIG. 5). From the Day 3H&E stain, it is clear that very few neutrophils have actually entered the mucosal layer and hence in the fluorescent section, only minimal infiltration of mucosal layer by QDs was observed. This indicates that at this point the disease is not severe with very little inflammation showing up. On Day 5 animals showed a measure decrease in their weight along with the presence of blood in stools. This indicates that an acute inflammatory process was already in progress in the colon. This is corroborated from the images below (FIG. 6) where there is increase in intensity of the QDs and QDs have labeled the whole mucosa. When compared to Day 3, the Day 5H&E stain shows increased count of infiltration by neutrophils. The crypts have shortened and DAI has increased indicating increase in disease severity.

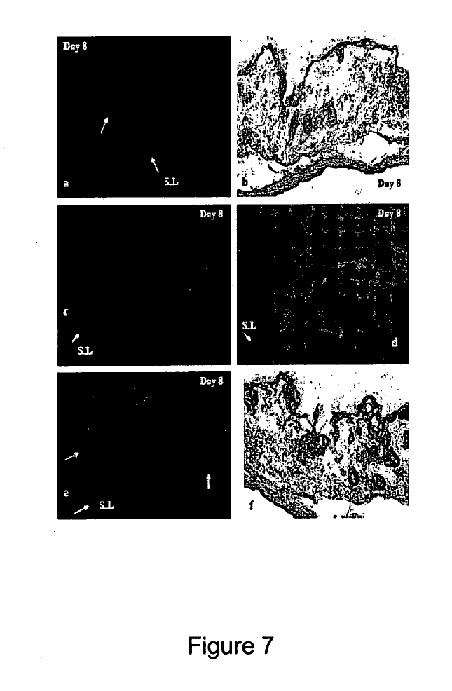

The images shown in FIG. 7 are taken from Day 8 of the DSS feed. The H&E stains show total loss of crypts with tissue destruction already evident in some locations. Consequently at this point the MPO level is high and hence there is increased in intensity of QDs as compared to previous days. As seen from the images the QDs have accumulated in the lamina propria of the tissue suggesting heavy release of myeloperoxidase.

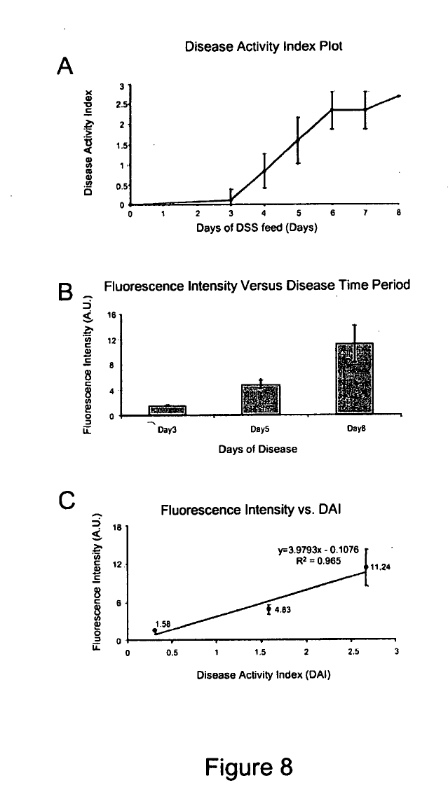

In this experiment for each animal multiple images were obtained and processed. The intensities from all the images were obtained for the particular day for all animals and were averaged This helped in mapping the disease along the colon and also made sure that the “patchiness” of the disease has been taken into account as observed in the initial days of the DSS feed. This process was done for all the three days. i.e. Day 3, Day 5 and Day 8, and the average values were plotted against the DAI score for that day. FIG. 8A shows the DAI plot for this experiment based on the clinical parameters monitored. The disease increases gradually over a period of time. FIG. 8B shows the fluorescence intensity plot of multiple images over the days of DSS feed vs. disease time period. The plot suggests a direct relationship between the intensity associated MPO expression level to DSS feed. Hence the fluorescence intensity was plotted against the DAI using DAI as a clinical index of disease progression. FIG. 8C establishes the correlation between the fluorescence intensity and the respective DAI values.

From the above graphs an excellent correlation can be observed between the disease severity and the fluorescence intensity values. The correlation values is almost the same to that observed in the earlier experiment thus demonstrating the reproducibility of the data. The graphs from these experiments demonstrated that the intensity of inflammation can be successfully quantified in terms of the fluorescence intensity values of the MPO-QD conjugates.

### Experiment #4

### Specificity of the Probe Designed in the DSS Model of Colitis

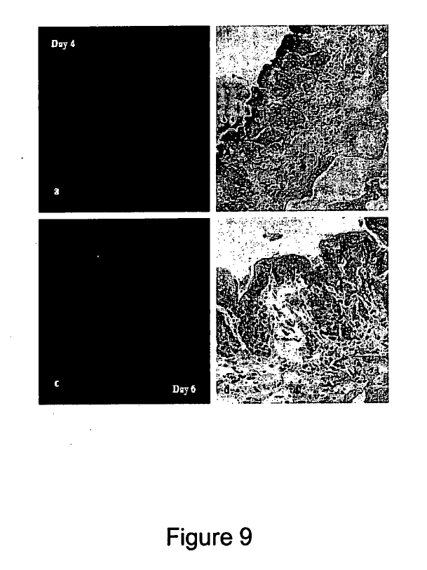

It is necessary to establish the specificity of the probe designed in in vivo conditions. To test the specificity, 655 QDs were conjugated to testosterone antibodies and used in the same model. Since testosterone antigens are never expressed in the GI tract the QDs should attach minimally to the tissue regardless of the inflammation level. FIG. 9 shows images form Day 4 and Day 6 along with the H&E stains.

The H&E stains from both days are in line with observations of disease progression in this model. Despite the observed shortening of the crypts and significant increase in neutrophils in the mucosal layer on Day 4, there are very few Quantum Dots in the tissue (FIG. 9A). A very small number of QDs observed in these images can only be attributed to QDs simply sticking to the surface of the tissue or being contained in tissue folds as compared to real targeting observed in the QD MPO conjugate images. On Day 6 (FIG. 9C) the QDs are localized to the exterior of the mucosal layer and there is complete absence of QDs in any other part. This clearly indicated that even though there is severe tissue destruction QDs were not sequestered in the tissue. This proves that the conjugate designed is specific and targets only the antigen/protein of interest, preferably MPO.

### Experiment #5

### Targeting Three Biomarkers IL1-α, MPO, and TNFα With Quantum Dots in the DSS Model of Colitis

The study was designed to test the visualization of multiple biomarkers using QD conjugates. In this experiment, three markers were targeted which are expressed in both acute and chronic stages of inflammation. IL1α and TNFα are important proinflammatory cytokines that initiate the recruitment of neutrophils, macrophages, as well as T cells and B cells. Their targeting could help in monitoring both phases of inflammation and also can help visualize how their localization changes with respect to disease progression.

Antibody to cytokine IL1α was conjugated to 605 nm QDs and antibody to TNFα was conjugated to a 705 nm QD. The emission wavelength windows were adjusted accordingly so as to have minimum overlap between the three markers. These QDs were chosen so as to avoid the autofluorescence of the tissue sections.

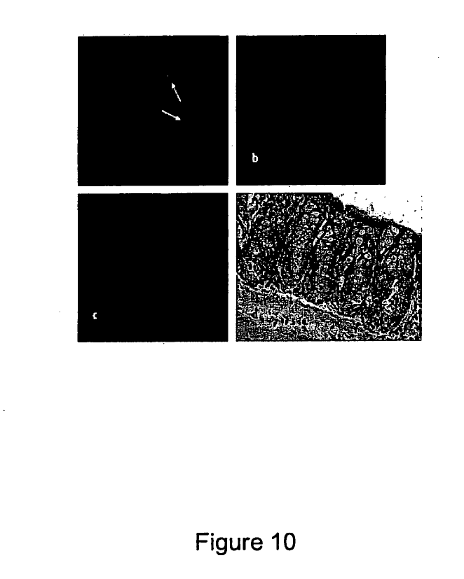

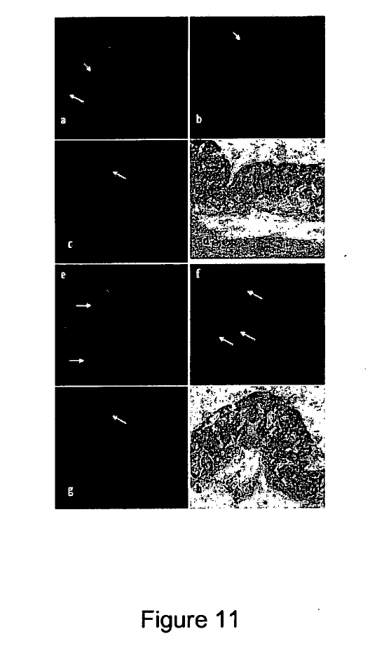

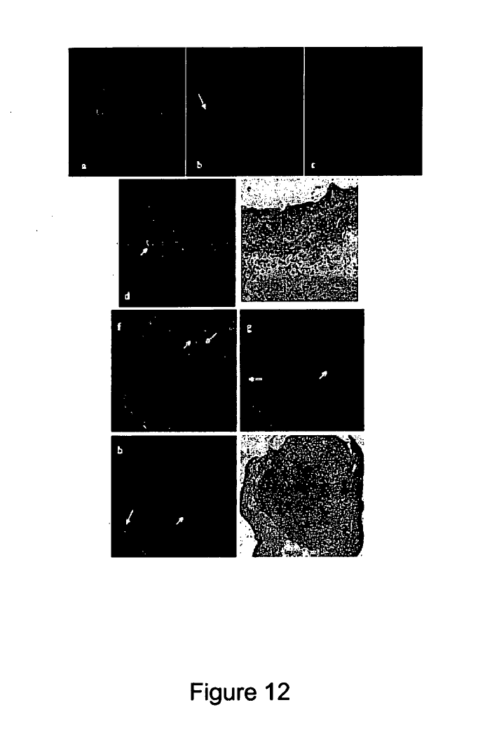

FIG. 10, FIG. 11 and FIG. 12 show the maximum projection images from Day 3, Day 6 and Day 7 in the acute stage of the experimental colitis. From the Day 3H&E (FIG. 10D) stain it is evident that there is no shortening of crypt layers and very few neutrophils are present in the tissue. From the fluorescent images, the presence of IL1α (FIG. 10A) was observed, but there was very little of MPO (FIG. 10B) whereas 705 QDs fails to target TNFα (FIG. 10C).

MPO levels of Day 3, Day 5 and Day 7 were in accordance with previous experiments. IL1α intensity increases from Day 3 and is highest Day 7. Similar inferences can be made for TNFα though it is clear that the TNFα intensity is not as strong as IL1α or MPO.

FIG. 12I shows an H&E stain from Day 7 with a heavy accumulation of inflammatory cells. Corresponding fluorescent images i.e. FIG. 12F, FIG. 12G, and FIG. 12H, show the presence of different markers at different sites as well as their colocalization.

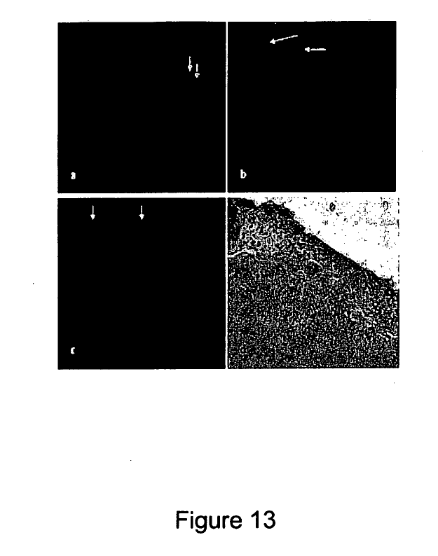

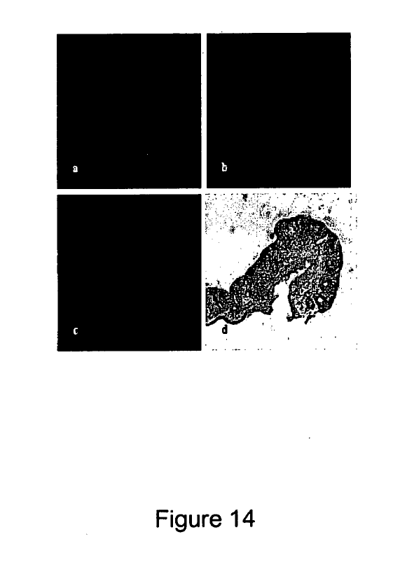

The experiment was also designed to study the expression of all these three markers during the chronic stage of inflammation (FIG. 13 and FIG. 14) as compared to the acute stage. The chronic stage is marked mainly by infiltration with monocytes, macrophages and intermittent infiltration with neutrophils but in much less amount. At the same time, tissue healing starts with regeneration of crypt structure that was lost during the acute inflammation period. Cytokines, especially TNFα, are responsible for recruitment of macrophages and monocytes and their activation and hence going into the chronic stage TNFα levels are expected to be highest as compared to all the markers.

On Day 14 (FIG. 13) when chronic inflammation has started there are very few neutrophils but abundant macrophages, monocytes and lymphocytes. These cell types carry out the simultaneous foreign particle destruction as well as tissue reconstruction. At this stage TNFα levels in the serum are significantly higher since they help in the recruitment of these cells. It has also been shown in earlier studies (Murthy et al., unpublished data) that IL1α and TNFα levels increase in the chronic stage of this model. The presence of MPO in some sections shows that there is intermittent acute inflammation going on. However as compared to the acute inflammatory stage the MPO expression level is not high.

Although these studies targeted three markers in both stages, two of the markers were not present in the chronic stage. In chronic inflammation, TNFα levels increase in serum; however QD conjugates failed to identify TNFα in the chronic stage. The level of IL1α decreases in the chronic stage and hence its level was not expected to be high on the 14^{th} (FIGS. 13) and 21^{st} (FIG. 14) day of the experiment by the QD conjugate.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of identifying an inflammatory condition in a mammal, said method comprising:
a. obtaining a biological sample from a mammal;
b. contacting said sample with a conjugate, wherein said conjugate comprises a reporter component and an antibody that specifically binds to a biomarker, wherein said reporter component is a quantum dot; and
c. determining whether said conjugate binds to said biomarker,
further wherein a binding of said conjugate to said biomarker is an indication that said mammal is afflicted with said inflammatory condition.

2. A method of identifying an inflammatory condition in a mammal, said method comprising:
a. obtaining a biological sample from a mammal;
b. contacting said sample with a conjugate, wherein said conjugate comprises a reporter component and an antibody that specifically binds to a biomarker, wherein said reporter component is at least one member selected from the group consisting of a magnetic nanoparticle and a magnetic quantum dot; and
c. determining whether said conjugate binds to said biomarker,
further wherein a binding of said conjugate to said biomarker is an indication that said mammal is afflicted with said inflammatory condition.

3. The method of claim 1, wherein said mammal is a human.

4. The method of claim 1, wherein the method comprises detecting two or more biomarkers in said biological sample.

5. The method of claim 1, wherein said biomarker is selected from the group consisting of an enzyme, an adhesion molecule, a cytokine, a protein, a lipid mediator, an immune response mediator, and a growth factor.

6. The method of claim 1, wherein said biomarker is selected from the group consisting of myeloperoxidase (MPO), IL1α, TNFα, perinuclear anti-neutrophil cytoplasmic antibody (p-ANCA), anti-Saccharomyces cerevisiae antibody (ASCA), angiotensin converting enzyme, lactoferrin, C-reactive protein (CRP), and calprotectin.

7. The method of claim 1, wherein said inflammatory condition comprises at least one inflammatory disease selected from the group consisting of inflammatory bowel disease, ulcerative colitis, Crohn's disease, stroke, myocarditis, cardiovascular disease, acute coronary syndromes, acute myocardial infarction, pericarditis, periodontal disease, cancer, Alzheimer's disease, and autoimmune diseases.

8. The method of claim 1, wherein said method comprises an immunoassay for detecting a biomarker in said sample.

9. The method of claim 8, wherein said immunoassay is selected from the group consisting of Western blot, ELISA, immunoprecipitation, immunohistochemistry, immunofluorescence, radioimmunoassay, dot blotting, and FACS.

10. The method of claim 1, wherein said method comprises a nucleic acid assay for detecting a nucleic acid encoding said biomarker in said sample.

11. The method of claim 10, wherein said nucleic assay is selected from the group consisting of a Northern blot, Southern blot, in situ hybridization, a PCR assay, an RT-PCR assay, a probe array, a gene chip, and a microarray.

12. A kit comprising:
a. a composition for detecting a biomarker in a biological sample obtained from a mammal, wherein said composition comprises at least one conjugate, further wherein said conjugate comprises a reporter component and an antibody that specifically binds to a biomarker, further wherein said reporter component is a quantum dot, and
b. instructional material for the use thereof.

13. A kit comprising:
a. a composition for detecting a biomarker in a biological sample obtained from a mammal, wherein said composition comprises at least one conjugate, further wherein said conjugate comprises a reporter component and an antibody that specifically binds to a biomarker, further wherein said reporter component is at least one member selected from the group consisting of a magnetic nanoparticle and a magnetic quantum dot, and
b. instructional material for the use thereof.

14. The kit of claim 12, wherein said mammal is a human.

15. The kit of claim 12, wherein said biomarker is selected from the group consisting of myeloperoxidase (MPO), IL1α, TNFα, perinuclear anti-neutrophil cytoplasmic antibody (p-ANCA), anti-Saccharomyces cerevisiae antibody (ASCA), angiotensin converting enzyme, lactoferrin, C-reactive protein (CRP), and calprotectin.

16. The kit of claim 12, wherein said antibody is bound to a substrate surface.
